(19) United States
(12) Patent Application Publication
(10) Pub. No.: US 2003/0186836 A1
(43) Pub. Date: Oct. 2, 2003

(54) Alcohol- free base for aqueous perfume composition, and alcohol -free aqueous perfume composition comprising same

(76) Inventors: Martine Dumanois, Grosbreuil (FR); Nathalie Gueyne, Paris (FR)

Correspondence Address: CASELLA & HESPOS, 274 MADISON AVENUE, NEW YORK, NY, 10016

(21) Appl. No.: 10/312,922

(22) PCT Filed: Jun. 29, 2001
(86) PCT No.: PCT/FR01/01994

(51) Int. Cl. A61K 7/46

(52) U.S. Cl. 512/2

(57) ABSTRACT

The present invention relates to a base for an alcohol-free, non-fatty, non-sticky perfumed aqueous cosmetic composition, including an aqueous medium, one or more non-ionic surfactants and a solvent, this base being characterized in that said solvent is isoprene glycol.

It also relates to the aforementioned cosmetic composition, including the above-mentioned base and one or more aromatic substances.

The present invention relates to a base for an alcohol-free, non-fatty, non-sticky perfumed aqueous cosmetic composition, such as a perfume, a toilet water, a perfumed gel or a deodorant water; it also relates to such a composition including such a base.

Hitherto, alcohol-free aqueous cosmetic compositions, the appearance of which can range from that of milk to that of a clear, transparent liquid, generally include the following components:

a mixture of natural and/or synthetic essential oils insoluble in water;

one or more cationic surfactants, one or more anionic surfactants, a mixture thereof, or one or more non-ionic surfactants; these surfactants, which are generally present in high doses, are intended, according to their content, to emulsify or to dissolve the mixture of essential oils;

a solubilising agent that is both lipophilic and hydrophilic, generally of the polyethylene glycol type;

an aqueous dispersing agent (generally water); and

one or more preservatives such as ethyl p-hydroxybenzoate (Nipagine^{R}) and propyl p-hydroxybenzoate (Nipasol^{R}).

Such known compositions, however, generally have the following drawbacks:

they are not always translucent;

they are sticky owing to the sticky nature of the surfactants and to the relatively large quantity of surfactants implemented;

the perfume given off by the essential oils is liable to be denatured by the solubilising agent;

they are somewhat irritant owing to the irritant nature of the solubilising agent and the aggressive nature of the surfactants; and

the essential oils are liable to be deteriorated by air oxidation, which affects the stability of the compositions and the olfactory characteristic thereof.

The object of the present invention is to eliminate the above drawbacks. The Applicant has thus sought for a means of reducing the dose of surfactants that are the origin, in particular, of the sticky and irritant nature of known cosmetic compositions.

It has now demonstrated that the object sought after could be achieved by using a specific solvent, isoprene glycol, in place of the known solubilising agents.

The present invention thus relates to a new base for an alcohol-free, non-fatty, non-sticky perfumed aqueous cosmetic composition, including an aqueous medium, one or more non-ionic surfactants and a solvent, this base being characterised in that said solvent is isoprene glycol.

The latter, which is both hydrophilic and lipophilic, and further known as 3-methyl 1,3-butanediol, makes it possible, in fact, thanks to its solvent and solubilising power, to very substantially reduce the quantity of surfactants to be used to arrive at a stable base and composition. This solvent further has characteristics that are absolutely essential in order to obtain a base suitable for use in cosmetics. Indeed, it enables translucent bases to be obtained; in addition, it does not denature the aromatic substance or substances intended for incorporation in said base in order to obtain a perfumed composition, and it is neither sticky, nor irritant, nor sensitive to air oxidation.

It will be noted that the surfactant or surfactants of the aforementioned base are preferably chosen from among the alcohols of ethoxylated fatty acids such as ethoxylated oleyl alcohol (for example oleyl alcohol ethoxylated with 10 moles of ethylene oxide, manufactured by the American company AMERCHOL and marketed in France under the AMEROXOLR trade mark), ethoxylated hydrogenated castor oil (for example, hydrogenated castor oil ethoxylated with 40 moles of ethylene oxide, marketed by the company BASF under the CREMOPHOR^{R} trade mark) and mixtures thereof.

It will be noted that the base according to the invention will preferably include a preservative such as NIPAGINER, for example.

Advantageously, the base includes isoprene glycol in a proportion of approximately 5 to 30% by weight, AMEROXOL^{R} in a proportion of approximately 0.5 to 5.0% by weight and CREMOPHOR^{R} in a proportion of approximately 0.5 to 5.0% by weight, the remainder being essentially purified water.

The present invention further relates to a perfumed aqueous cosmetic composition including a base as defined hereabove and one or more aromatic substances such as natural and/or synthetic essential oils.

This composition will advantageously include approximately 1-10% by weight of such aromatic substances.

According to one preferred form of embodiment, said perfumed composition includes the following components:

| | | |
|---|---|---|
| essential oils | 1.0-6.0 | g |
| isoprene glycol | 5.0-20.0 | g |
| CREMOPHOR ® | 0.5-3.5 | g |
| AMEROXOL ® | 0.5-3.5 | g |
| NIPAGINE ® | 0.1-0.2 | g |
| purified water q.s. for | 100.0 | g |

The base and composition according to the invention can, for example, be prepared using the following process.

The preservative or preservatives (for example, NIPAGINE^{R}) is/are solubilised in the isoprene glycol and the aqueous medium (purified water) is added to the solution thus obtained to obtain a first phase.

Separately, the surfactants are warmed to cause them to melt, the aromatic substances are added thereto and homogenisation is carried out to obtain a second phase.

Finally, the first phase is added to this second phase slowly and with stirring.

Two examples of a preferred aqueous cosmetic composition according to the invention are given below by way of non-limitative illustration.

### EXAMPLE 1

### Perfume Water

This water has the following composition by weight:

| | | |
|---|---|---|
| essential oils | 5.0 | g |
| isoprene glycol | 15.0 | g |
| CREMOPHOR ® | 2.5 | g |
| AMEROXOL ® | 2.5 | g |
| NIPAGINE ® | 0.15 | g |
| purified water q.s. for | 100.0 | g |

### EXAMPLE 2

### Toilet Water

| | | |
|---|---|---|
| essential oils | 1.0 | g |
| isoprene glycol | 5.0 | g |
| CREMOPHOR ® | 1.0 | g |
| AMEROXOL ® | 0.5 | g |
| NIPAGINE ® | 0.15 | g |
| purified water q.s. for | 100.0 | g |

1. Base for an alcohol-free, non-fatty, non-sticky perfumed aqueous cosmetic composition, including an aqueous medium, one or more non-ionic surfactants and a solvent, this base being characterized in that said solvent is isoprene glycol.

2. Base according to claim 1, characterized in that the surfactant or surfactants is/are chosen from among the alcohols of ethoxylated fatty acids, ethoxylated hydrogenated castor oil and mixtures thereof.

3. Base according to claim 2, characterized in that the surfactant or surfactants is/are chosen from among oleyl alcohol ethoxylated with 10 moles of ethylene oxide, hydrogenated castor oil ethoxylated with 40 moles of ethylene oxide and mixtures thereof.

4. Base according to claim 3, characterized in that it includes 5 to 30% by weight of isoprene glycol, 0.5 to 5.0% by weight of oleyl alcohol ethoxylated with 10 moles of ethylene oxide and 0.5 to 5.0% by weight of hydrogenated castor oil ethoxylated with 40 moles of ethylene oxide.

5. Non-fatty, non-sticky perfumed aqueous cosmetic composition, characterized in that it includes a base according to one of preceding claims and one or more aromatic substances.

6. Composition according to claim 5, characterized in that said aromatic substances are natural and/or synthetic essential oils.

7. Composition according to claim 5 or 6, characterized in that it includes 1 to 10% by weight of aromatic substances.

8. Composition according to claim 5, 6 or 7, characterized in that it includes the following components:

| | | |
|---|---|---|
| essential oils | 1.0-6.0 | g |
| isoprene glycol | 5.0-20.0 | g |
| CREMOPHOR ® | 0.5-3.5 | g |
| AMEROXOL ® | 0.5-3.5 | g |
| NIPAGINE ® | 0.1-0.2 | g |
| purified water q.s. for | 100.0 | g |

9. Composition according to claim 8, characterized in that it includes the following components:

| | | |
|---|---|---|
| essential oils | 5.0 | g |
| isoprene glycol | 15.0 | g |
| CREMOPHOR ® | 2.5 | g |
| AMEROXOL ® | 2.5 | g |
| NIPAGINE ® | 0.15 | g |
| purified water q.s. for | 100.0 | g |

10. Composition according to claim 8, characterized in that it includes the following components:

| | | |
|---|---|---|
| essential oils | 1.0 | g |
| isoprene glycol | 5.0 | g |
| CREMOPHOR ® | 1.0 | g |
| AMEROXOL ® | 0.5 | g |
| NIPAGINE ® | 0.15 | g |
| purified water q.s. for | 100.0 | g |
